(19) United States
(12) Patent Application Publication
(10) Pub. No.: US 2023/0394988 A1
(43) Pub. Date: Dec. 7, 2023

(54) METHODS AND SYSTEMS FOR CONTINUOUS MONITORING OF TASK PERFORMANCE

(71) Applicant: THE ROYAL INSTITUTION FOR THE ADVANCEMENT OF LEARNING/MCGILL UNIVERSITY, Montréal (CA)

(72) Inventors: Muhammed Recai YILMAZ, Montreal (CA); Alexander WINKLER-SCHWARTZ, Montreal (CA); Nykan MIRCHI, Beaconsfield (CA); Rolando Fausto DEL MAESTRO, Tiverton (CA)

(21) Appl. No.: 18/031,959

(22) PCT Filed: Oct. 14, 2021
(86) PCT No.: PCT/CA2021/051440

§ 371 Date: Apr. 14, 2023

Related U.S. Application Data
(60) Provisional application No. 63/091,629, filed on Oct. 14, 2020.

(51) Int. Cl. G09B 19/00; G09B 9/00; G06T 19/00

(52) U.S. Cl. CPC G09B 19/00; G09B 9/00; G06T 19/003; G06T 2219/024

(57) ABSTRACT

There are described a method and system for obtaining data at a plurality of time intervals throughout a task performed by a user, the data generated by a control device manipulated by the user while performing the task; determining at least one task metric from the data, the at least one task metric associated with the task; using the at least one task matric to assign a value to at least one quality assessment metric at each time interval throughout the task based on a progression curve having a novice skill level at a first end of the curve, an expert skill level at a second end of the curve opposite to the first end, and undefined skill levels in between, the at least one quality assessment metric associated with the task; and displaying in real-time a first time-varying graphical indicator indicative of the value of the at least one quality assessment metric.

### CROSS-REFERENCE TO RELATED APPLICATIONS

The present application claims the benefit of U.S. Provisional Patent Application No. 63/091,629 filed on Oct. 14, 2020, the contents of which are hereby incorporated by reference.

### TECHNICAL FIELD

The disclosure generally relates to the field of technical skills training and/or assessment devices.

### BACKGROUND OF THE ART

There is no gold standard for assessing human performance in many complex fields such as surgery. Present methods depend on apprenticeship models where trainees are evaluated by experts. These methods may be combined and/or enhanced with intelligent computer systems, such as virtual reality simulators, that offer various features, such as visual guidance and haptic feedback. While the current intelligent computer systems for technical skills training and assessment are suitable for their purposes, improvements are desired.

### SUMMARY

In accordance with one aspect, there is provided a method comprising obtaining data at a plurality of time intervals throughout a task performed by a user, the data generated by a control device manipulated by the user while performing the task, determining at least one task metric from the data, the task metric associated with the task, using the at least one task metric to assign a value to at least one quality assessment metric at each time interval throughout the task based on a progression curve having a novice skill level at a first end of the curve, an expert skill level at a second end of the curve opposite to the first end, and undefined skill levels in between, the at least one quality assessment metric associated with the task, and displaying in real-time a first time-varying graphical indicator indicative of the value of the at least one quality assessment metric.

In some embodiments, the method further comprises assigning, at the computing device, a value to at least one risk metric from the data, the at least one risk metric indicative of a negative outcome associated with the task and displaying in real time, at the computing device, a second time-varying graphical indicator indicative of the value of the at least one risk metric.

In some embodiments, the method further comprises displaying, at the computing device, a graphical warning to the user when the value of the at least one risk metric reaches a first threshold.

In some embodiments, the method further comprises predicting, at the computing device, the value of the at least one task metric for the expert skill level at a current time point in the task and providing, at the computing device, guidance to the user in real-time based on a difference between the predicted value and an actual value of the at least one task metric.

In some embodiments, providing guidance to the user in real-time comprises displaying a guidance message associated with improving the value of the at least one task metric.

In some embodiments, the guidance message is displayed when the difference between the predicted value and the actual value of the at least one task metric is above a second threshold.

In some embodiments, assigning the value to the at least one quality assessment metric comprises applying a regression model that scores the at least one quality assessment metric at every time interval.

In some embodiments, the method further comprises using a recurrent neural network to implement the regression model.

In some embodiments, displaying in real-time the first time-varying graphical indicator comprises presenting the first time-varying graphical indicator adjacent to a skill level scale representative of the progression curve.

In some embodiments, the task is performed by the user on a virtual simulator.

In accordance with another aspect, there is provided a system comprising a processing unit and a non-transitory computer-readable medium having stored thereon program instructions. The program instructions are executable by the processing unit for obtaining data at a plurality of time intervals throughout a task performed by a user, the data generated by a control device manipulated by the user while performing the task, determining at least one task metric from the data, the task metric associated with the task, using the at least one task metric to assign a value to at least one quality assessment metric at each time interval throughout the task based on a progression curve having a novice skill level at a first end of the curve, an expert skill level at a second end of the curve opposite to the first end, and undefined skill levels in between, the at least one quality assessment metric associated with the task; and displaying in real-time a first time-varying graphical indicator indicative of the value of the at least one quality assessment metric.

In some embodiments, the program instructions are further executable for assigning a value to at least one risk metric from the data, the at least one risk metric indicative of a negative outcome associated with the task, and displaying in real time a second time-varying graphical indicator indicative of the value of the at least one risk metric.

In some embodiments, the program instructions are further executable for displaying a graphical warning to the user when the value of the at least one risk metric reaches a first threshold.

In some embodiments, the program instructions are further executable for predicting the value of the at least one task metric for the expert skill level at a current time point in the task, and providing guidance to the user in real-time based on a difference between the predicted value and an actual value of the at least one task metric.

In some embodiments, providing guidance to the user in real-time comprises displaying a guidance message associated with improving the value of the at least one task metric.

In some embodiments, the program instructions are executable for displaying the guidance message when the difference between the predicted value and the actual value of the at least one task metric is above a second threshold.

In some embodiments, assigning the value to the at least one quality assessment metric comprises applying a regression model that scores the at least one quality assessment metric at every time interval.

In some embodiments, a recurrent neural network is used to implement the regression model.

In some embodiments, displaying in real-time the first time-varying graphical indicator comprises presenting the first time-varying graphical indicator adjacent to a skill level scale representative of the progression curve.

In some embodiments, the program instructions are executable for obtaining the data throughout the task performed by the user on a virtual simulator.

Many further features and combinations thereof concerning embodiments described herein will appear to those skilled in the art following a reading of the instant disclosure.

### DESCRIPTION OF THE FIGURES

Reference is now made to the accompanying figures in which:

FIG. 1 is a block diagram of an example simulation system;

FIG. 2 is a schematic of an example progression curve;

FIG. 3A is a schematic representing training of an algorithm;

FIG. 3B is a schematic diagram representing an example neural network;

FIGS. 4A-4B are examples of heat maps of an expert performance and a novice performance, respectively, for a task;

FIGS. 5A-5C are flowcharts of example methods for operating the simulation system of FIG. 1,

FIGS. 6A-6C are screenshots of an example simulated scenario with task metrics and risk metrics displayed; and

FIG. 7 is a block diagram of an example computer device.

### DETAILED DESCRIPTION

There are described herein a deep learning-based simulation system and method which can continuously monitor human performance to enhance skillsets, with the capacity to alert the user of high risk behavior when error is imminent. The method can be applied to any real time performance data where the quality of performance can be labeled. The methods can be applied to any data providing instrument. Therefore, performance is continuously monitored, concrete suggestions can be made to improve performance, and error avoidance measures can function to alert operators of high risk behavior and imminent error.

The system provides a comprehensive and detailed assessment concerning continuous skills necessary for expert performance, combined with error detection and avoidance, and coaching. The tool can work with multiple algorithms simultaneously. One algorithm can make the quality assessment, recognizing the expertise of the performer during the performance. A second algorithm can detect the errors and assess risk. A third algorithm can make suggestions during the procedure to improve user performance. The system therefore has flexibility depending on the need outlined by the user. It is broadly applicable where data can be labeled with the known performance quality. A plurality of time points are labeled with a quality, so that the algorithm recognizes the quality during the flow of the performance. By modifying the output of the algorithm, the tool can give suggestions to improve performance and warnings of high-risk behavior and imminent error during performance. The tool can therefore assess the performance quality on a continuous basis, guide skill sets through coaching, and provide a mechanism to assess and alert the user of issues which may result in imminent error.

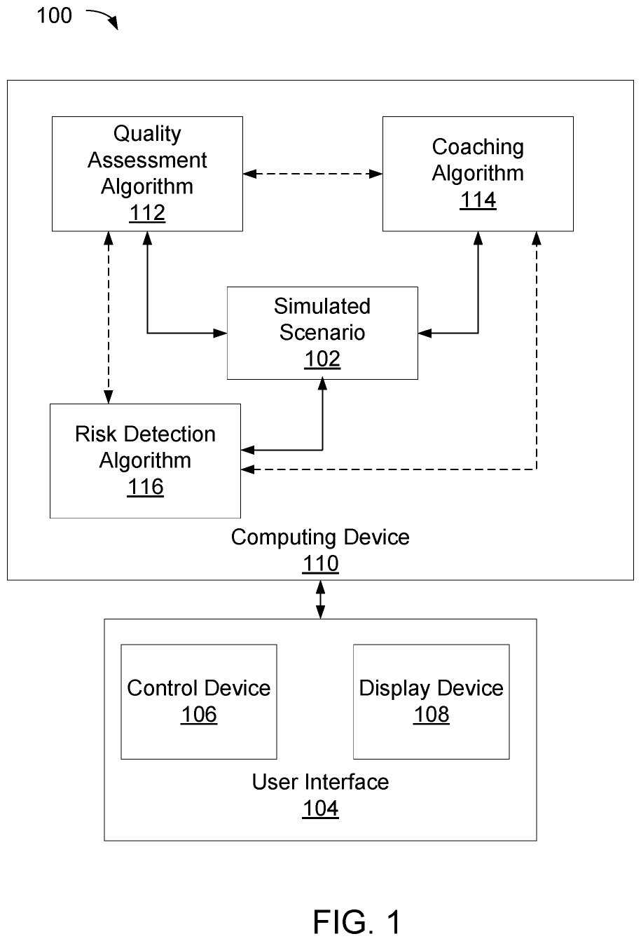

FIG. 1 shows an example of a simulation system 100, in accordance with some embodiments. The system 100 provides a simulated scenario 102 via a computing device 110. In one embodiment, the simulated scenario 102 comprises a virtual environment and at least one task to be completed in the virtual environment. It should however be understood that other embodiments may apply and the at least one task may be completed in an environment other than a virtual environment. For example, the task may be a real-life bimanual task completed in a real-world physical environment. A user performs one or more actions to complete the task. For example, the simulated scenario 102 may be of a medical procedure. Accordingly, the system 100 may be a medical or surgical simulation system. By way of another example, the simulated scenario 102 may be of a driving course. Accordingly, the system 100 may be a driving simulator.

In some embodiments, the system 100 may be a virtual reality system, whereby the user is immersed in the virtual world and experiences various sensory feedbacks. As previously noted, embodiments other than a virtual reality system may apply. The computing device 100 implements an application for providing the simulated scenario 102 that the user interacts with via a user interface 104. The user interface 104 comprises at least one control device 106 and at least one display device 108 for the user to interact with the simulated scenario 102. For example, the control device 106 may be a medical tool that is used during a medical procedure and virtual reality images are displayed on the display device 108. In another example, the control device 106 may comprise a steering wheel, an accelerator and a break, and a driving course is displayed on the display device 108. The display device 108 may be a display screen, a 3-dimensional headset/goggles or any other suitable visual interface. As the control device 106 is moved by the user, a graphical representation of the control device 106 may correspondingly move within a graphical environment of the simulated scenario 102 on the display device 108. The control device 106 may comprise one or more sensors for monitoring the movement of the control device 106. In some embodiments, one or more sensors external to the control device 106 may be used for monitoring movement of the control device 106. The computing device 110 may be configured to record the measurements from the sensors and/or the user's interactions with the simulated scenario 102. The control device 106 may also provide users with haptic feedback relating the motions and interactions occurring during the simulated task.

Algorithms can be created for different purposes, depending on a chosen output metric (or metric of interest). In the example of FIG. 1, there is illustrated a quality assessment algorithm 112, a coaching algorithm 114, and a risk detection algorithm 116. In some embodiments, multiple ones of the algorithms 112, 114, 116 are run concurrently, depending on the features of the simulation system 100 that are enabled. The features may be enabled by a user or by another system operator.

The quality assessment algorithm 112 provides the ability to recognize and quantify the performance of the user. In some embodiments, the metric of interest in quality assessment 112 is an expertise level. The trained algorithm can recognize patterns within the performance, assess the quality continuously, and locate the time points of excellent and poor performance.

The coaching algorithm 114 provides guidance to the user in real-time. In some embodiments, the metric of interest for coaching is a performance feature, and the algorithm 114 learns how patterns relate to that particular metric considering the user's expertise level. Predictions can then be made to guide the user, outlining how an expert does the procedure in the same situation.

The risk detection algorithm 116 evaluates potential risks in real time and can warn the user to avoid errors. In some embodiments, the metric of interest for risk detection is a safety feature, and the algorithm is trained to recognize patterns related to that safety feature and identify performance patterns consistent with a risk of error. The trained algorithm may then predict the expected risk level for any point of time during the procedure. High and low risk periods during the performance can be identified and tracked.

Each algorithm is trained using data, and any data providing continuous output of human performance can be used. Data generated by the control device 106 as it is manipulated by the user while performing the task is obtained at a plurality of time intervals throughout the task. For example, raw data may be collected every 20 ms, 40 ms, 60 ms, etc. Data may be acquired using larger or smaller time intervals, as needed for a given application. Data may be acquired with a steady time interval or interpolated to provide information in uniform intervals. The time interval utilized should be short enough to capture all relevant data, but not too short since this will increase the computational burden for the algorithm training, and large time intervals may result in loss of important information. Defining an ideal time interval helps reduce data size, decrease the computer work required for analysis, and increase the interpretability of the results. For example, the 20 ms time interval, which represents 50 data points per second, may be converted to an average time frame of every 200 ms, which represents 5 data points per second.

In some embodiments, the data is normalized. Normalization is used to rescale all the metrics. For example, a z-score normalization method may be used, scaling all variables with their standard deviation. In other words, if a value is 0.7 standard deviations below the mean, this value will be represented by −0.7. Other normalization methods may also be used.

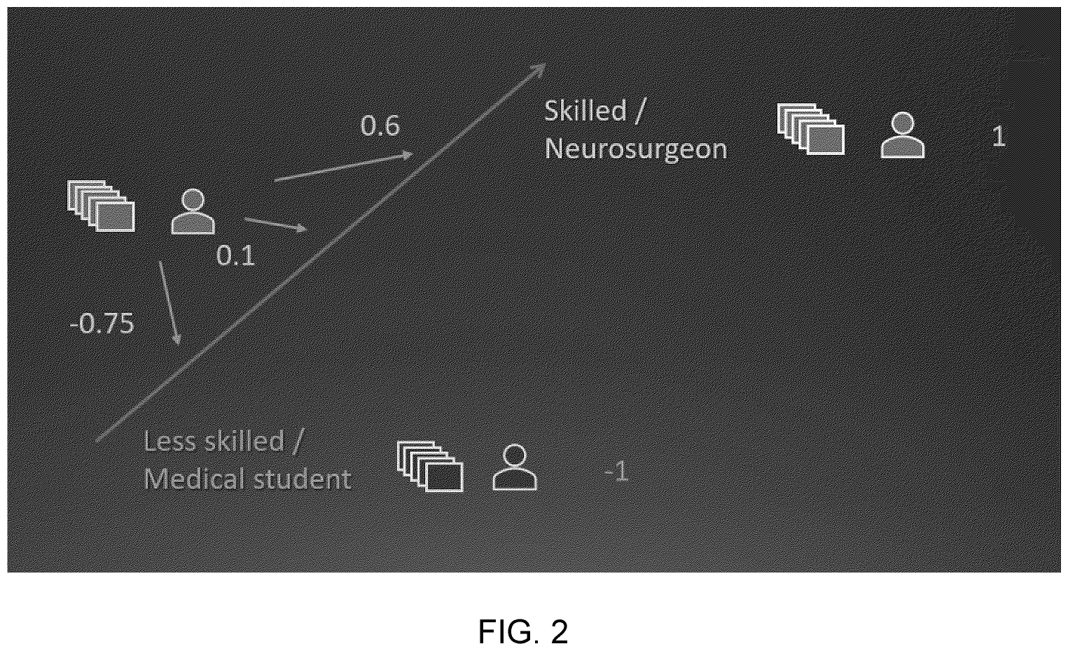

To train the algorithms, data from at least two expertise levels are used. An underlying assumption in the training of the algorithm(s) is that the learning of technical skills throughout the years follows a linear progression. An example is shown in FIG. 2, where a progression curve is defined between a first end point corresponding to a novice skill level and a second end point corresponding to an expert skill level. As used herein, the term “novice” refers to a person who is new (or beginner) to a given field or work in which they are placed and therefore has little experience, skill, and/or knowledge in the field or work. The novice is a person whose knowledge and skill level is not sufficient to allow them to perform at an acceptable standard. In order for a novice to become productive, the novice is taught (or shown) the basics of what is to be done. The term “expert” refers to a person with extensive experience, skill, and/or knowledge in the field or work. An expert has an advanced skill level that allows them to teach others (e.g., novices) what is to be done. Training the two ends of skill level allows the assessment of any skill level found in between the two ends, even undefined skill levels. Indeed, there is no need to provide pre-categorized expertise levels between the two end skill levels in order for the system 100 to recognize skill levels that fall between the two ends.

The expertise of the user can be used to label each time interval of a performance. The expertise is a class rather than a continuous variable. However, for the method, the objective is to create a regression model where each metric is composed of a numerical value. In this manner, the predictions of the algorithm are not classes, they are numerical values which represent the quality of the performance within a continuous scale. Therefore, the expertise classes are converted into numerical values. The highest level of expertise can be labeled with number ‘1’, representing one standard deviation above the mean quality, and the lowest end can be labeled with number ‘−1’, representing one standard deviation below the mean quality.

Using the expertise level to label each time point will not always result in correct labeling since a novice can perform at an expert level during some portions of the procedure being assessed. An expert can also perform at a novice level during some portions of the procedure. While this may be considered as a type of mislabeling, the quantity of the data will compensate for this issue. The algorithm will be able to differentiate the quality of performance of different expertise levels. In essence, an expert may be permitted to perform a number of movements which would be classified as non-expert, with the inverse also holding true.

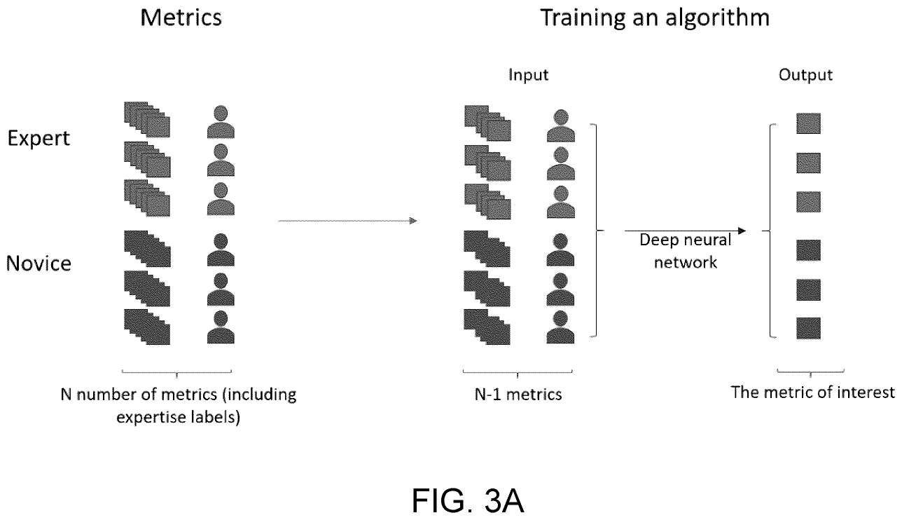

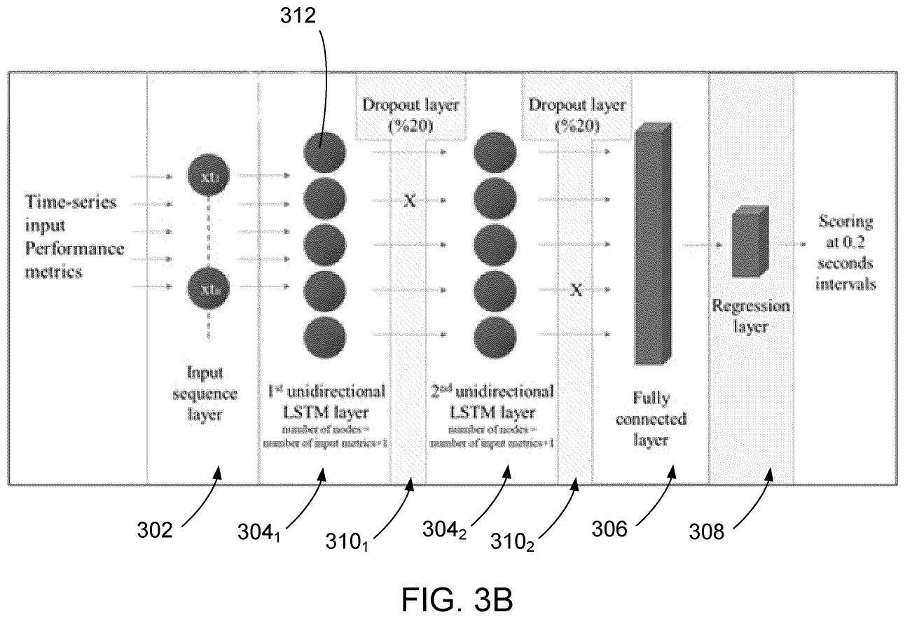

In some embodiments, metrics are extracted from raw data collected through the control device 106. As shown in FIG. 3A, metrics are extracted from data obtained from users at a novice level and users at an expert level. Metrics which are relevant to the task and critical to performance are created through a step of extracting metrics from the data. The metrics are calculated for each time interval and used to train the algorithms. When assessing the performance of a user, the algorithms are expected to recognize the values of the metric of interest at each time point and then be able to predict these values as accurately as possible. Root-mean-squared error can be used as a measure of how much the predictions deviated from the actual values. In some embodiments, the system recognizes the metrics and there is no metric extraction per se. In this case, raw data may be used directly.

Three datasets may be used for training: a training dataset, a validation dataset, and a testing dataset. The entire dataset can be split into three subsets (e.g. 70%, 15% and 15%) randomly. The training dataset is initially used for the algorithms to learn the data. The validation dataset is used to check the state of the training, for example to avoid overfitting of the data. The testing dataset can be used to evaluate the success of the training. Root-mean-squared errors for the three datasets should be close to each other and as close as possible to zero. Training can be repeated multiple times, optimizing hyper parameters and the structure of the algorithms, until the algorithms with the best root-mean-squared error results can be chosen as the final algorithms to be utilized.

In some embodiments, the algorithms used for continuous assessment of human performance are based on recurrent neural networks. These types of neural networks consider the relation between the data points, i.e. time points, allowing the algorithms to recognize the patterns within the performance as a whole within the flow. In some embodiments, long short-term memory (LSTM) networks are utilized, since they are designed to consider long term relations within the data. As opposed to other algorithms, long short-term memory networks can work on data with different lengths, suiting any length of performance or task. The LSTM network may implement the regression model used to score each metric at each time interval. In some embodiments, the LSTM network may be designed to minimize computational burden. As illustrated in FIG. 3B, the algorithms used for continuous assessment of human performance may be implemented using an LSTM network comprising an input sequence layer 302 (configured to receive as input time-series data of the user's interaction, e.g., in the virtual environment and performance metrics, i.e. metrics relevant to the task and critical to performance), two unidirectional LSTM layers 304₁ and 304₂, a fully connected layer 306, and a regression layer 308. Two dropout layers 310₁, 310₂ may be used after each LSTM layer to avoid data overfitting. The number of nodes as in 312 used for the LSTM layers may be calculated by adding one (1) to the number of input metrics (i.e. metrics relevant to the task and critical to performance). Sequence-to-sequence supervised learning may be used. Other embodiments may apply.

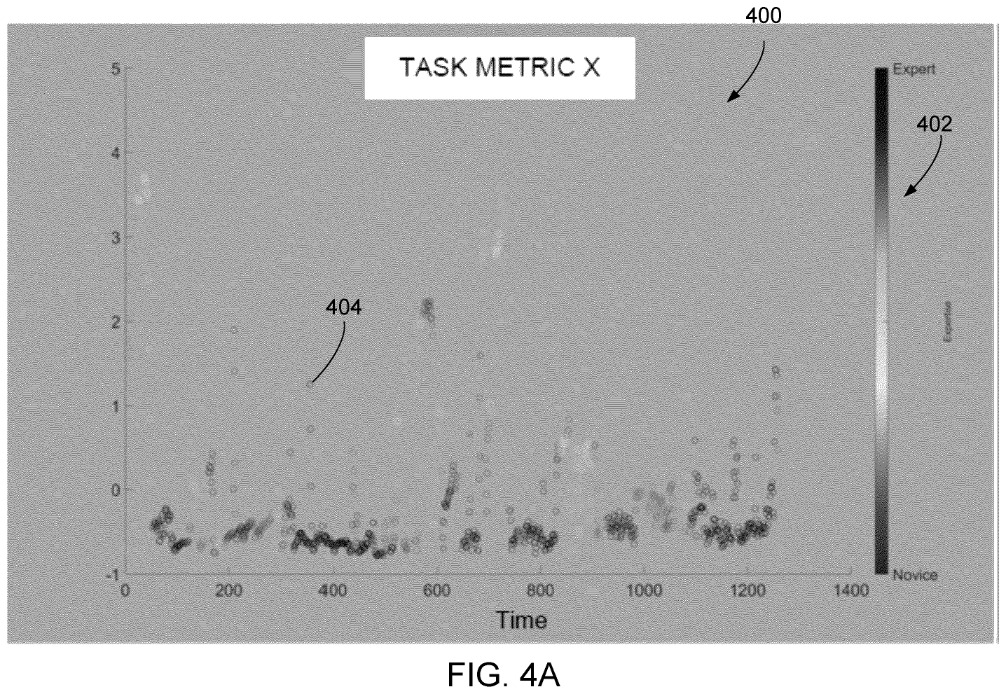

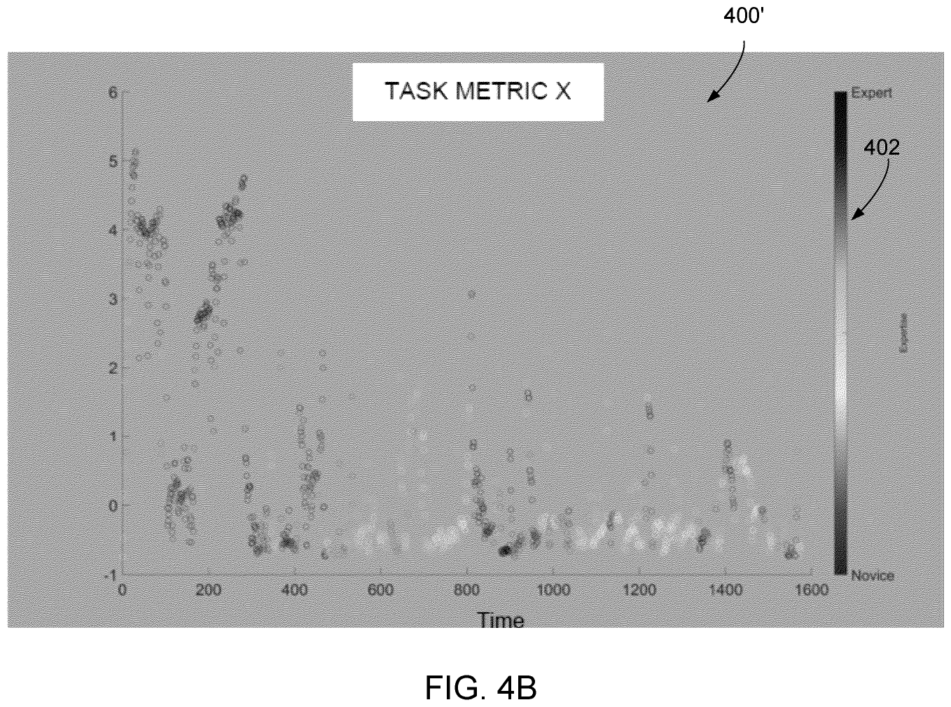

FIGS. 4A-4B illustrate example heat maps (also referred to as scatter plots) 400, 400′ of a given metric (task metric X) for an expert (FIG. 4A) and a novice (FIG. 4B). As shown, each point in time (the x-axis) has a numerical value or z-score (y-axis) associated therewith. A skill level scale 402 is shown on the right side of the graphs. In the illustrated embodiment, the skill level scale 402 represents the assessment made from skilled to less skilled levels of expertise. The maps 400, 400′ represent how the expertise assessment relates to the corresponding metric. Each dot as in 404 (FIG. 4A) represents an expertise assessment made at each time interval. In this example, the time intervals are 0.2 seconds and the x-axis indicates the number of decisions made by the user during the task.

With the trained algorithms, predictions can be made for any new data. If data is provided in real time, the system can provide predictions in real time, during the actual task. Therefore, a given metric of interest predicted by the trained algorithm to have a propensity for errors (high risk behavior) can be relayed to the user, who can then take corrective action by modifying his or her movements. This may be referred to as an error avoidance system, as it allows for a modification of performance before the error occurs. Verbal suggestions based on known expert behavior and expert video recordings can be provided during the procedure so the user has the opportunity to learn and thus continually modify his or her performance as necessary to obtain both an optimal and safe outcome.

Data is not always available in real time and pertinent data can be provided after task completion. In this situation, a video assessment of the user's performance can be displayed, outlining an assessment of his or her skills. Suggestions on improvements can be delineated and the user alerted to performance patterns which are considered high risk behavior.

Using predictions, the algorithms become capable of representing the experts' skill set. The error avoidance system should be flexible, allowing a range of choices in the performance of a specific task. However, it should be capable of identifying possible high risk behavior and outlining this pertinent information to the user. In some embodiments, a standard deviation range is defined and suggestions and/or warnings are output if the performance is outside of this range.

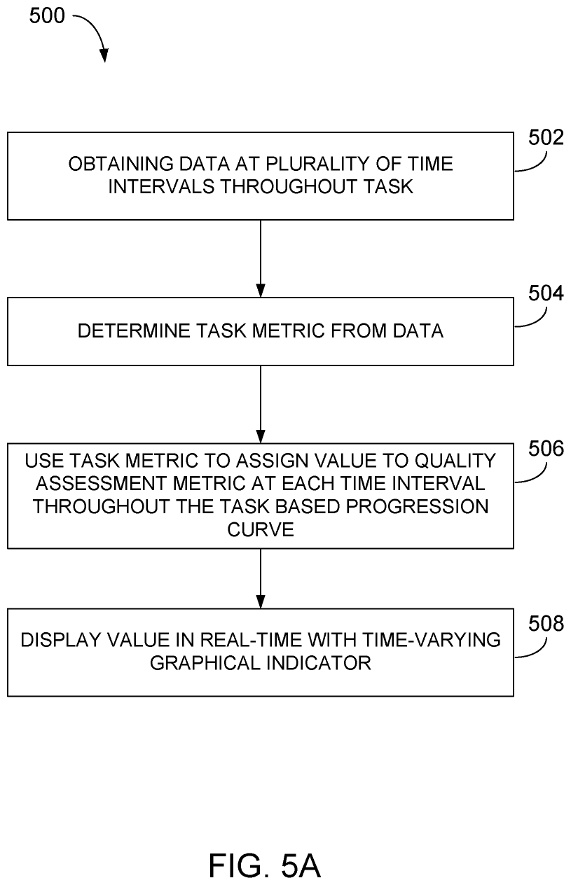

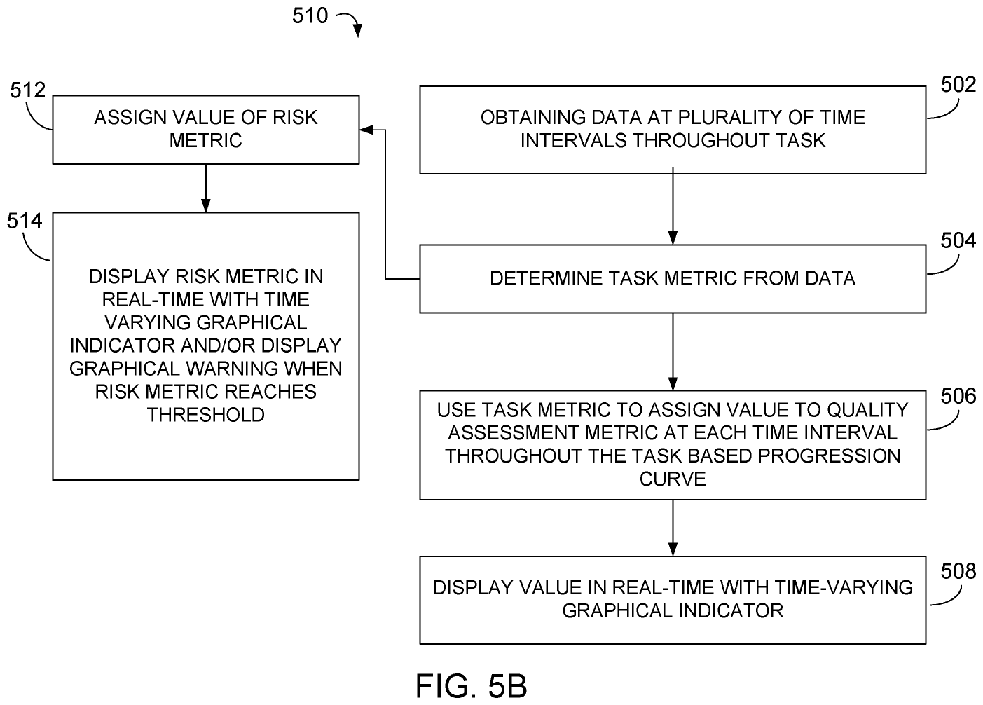

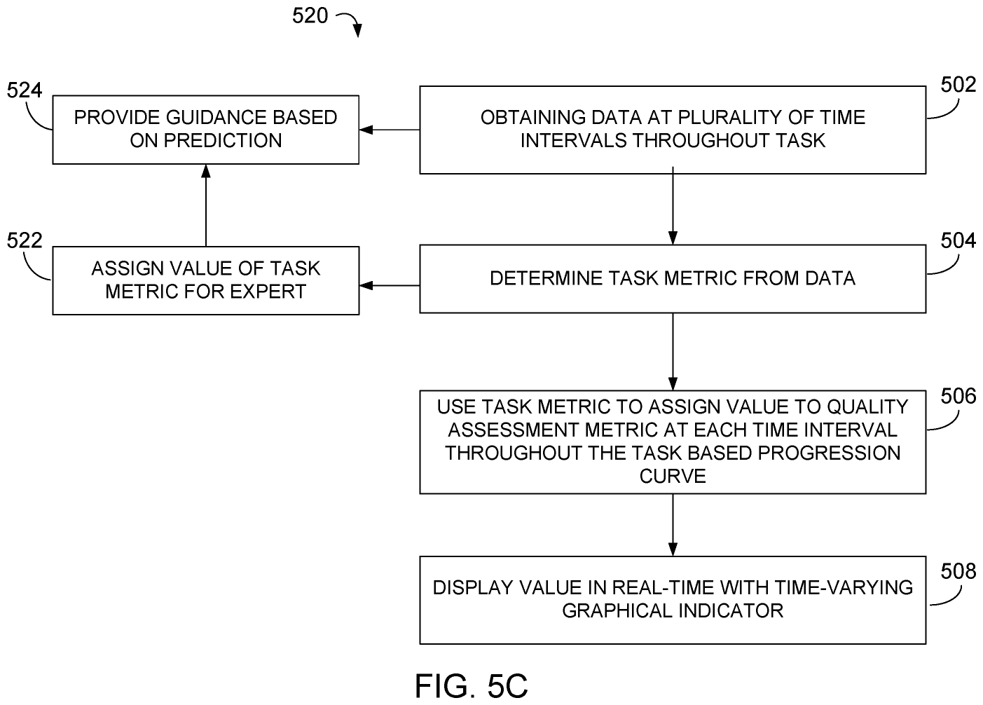

With reference to FIGS. 5A-5C, there are illustrated examples of a method for operating the simulation system 100, in accordance with various embodiments. FIG. 5A is an example of a method 500 with only a quality assessment feature enabled. At step 502, data is obtained at a plurality of time intervals throughout a task performed by a user on the simulated scenario 102. At step 504, at least one task metric is determined from the data, the task metric associated with the task and having a value. At step 506, using the at least one task metric determined at step 504, at least one quality assessment metric is assigned a score, or a value, by the algorithm at each time interval throughout the task. In one embodiment, the quality assessment metric (i.e. the metric of interest in quality assessment) is an expertise level, as described herein above. The value of the quality assessment metric is based on a progression curve having a novice skill level at the bottom end and an expert skill level at the top end, and undefined skill levels in between. An example of the progression curve is shown in FIG. 2. At step 508, the value for the quality assessment metric is displayed in real-time to the user. Since the value can change with each time interval, the value is displayed with a time-varying graphical indicator, such as a bar or line that gets longer or shorter proportionally to the value, and/or changes color with the value. The actual numerical value may also be displayed as the time-varying graphical indicator. Any graphical indicator that can vary proportionally with the changes to the value can be used to keep the user informed of his or her performance at any point in time.

FIG. 5B is an example of a method 510, with both a quality assessment feature and a risk detection feature enabled. At least one task metric determined at step 504 is used to assign a value to a risk metric by the algorithm, at each time interval, as per step 512. The risk metric is indicative of a negative outcome associated with the task. As used herein, “assigning a value” to a metric, whether a task metric or a risk metric for instance, refers to the algorithm assigning a value based on a prediction for that value. For the risk metric, a prediction may be made for the known skill level of the user, or for an expert skill level if the skill level of the user is unknown. The risk metric may be displayed to the user in real-time at step 514, using a time-varying graphical indicator. The time-varying graphical indicator for the risk metric may be similar to the time-varying graphical indicator for the task metric, but sufficiently different for the user to recognize the difference between the two. In some embodiments, a warning is provided to the user when the risk metric reaches a threshold (referred to herein as a first threshold). The warning may be visual, audio, and/or through haptic feedback of the control device.

FIG. 5C is an example of a method 520 with the quality assessment feature and a coaching feature enabled. At least one task metric determined at step 504 is used to assign a value of the task metric for an expert skill level, at each time interval, as per step 522. Step 522 may comprise assigning the value of the task metric based on a prediction made for the expert skill level, rather than for the skill level of the user. At step 524, guidance is provided to the user in real-time based on a difference between the assigned value of the task metric for an expert skill level and an actual value of the task metric, which may be determined from data obtained at step 502. The difference may be computed using any suitable technique including, but not limited to, standard deviation. A guidance message may be provided to the user when the difference is above a second threshold (different from the first threshold described above), the guidance message being associated with reducing the risk metric. The guidance message may allow the user to bring the actual value of the task metric closer to the assigned value of the task metric for the expert skill level. Guidance may be provided using various mechanisms, such as displaying a message associated with improving the task metric and/or reducing the risk metric, audio messages, and other forms of coaching (via any suitable output device) to allow the user to enhance his or her skills.

It will be understood that various combinations of the quality assessment feature, coaching feature, and risk detection feature may be provided, including having all three features enabled concurrently.

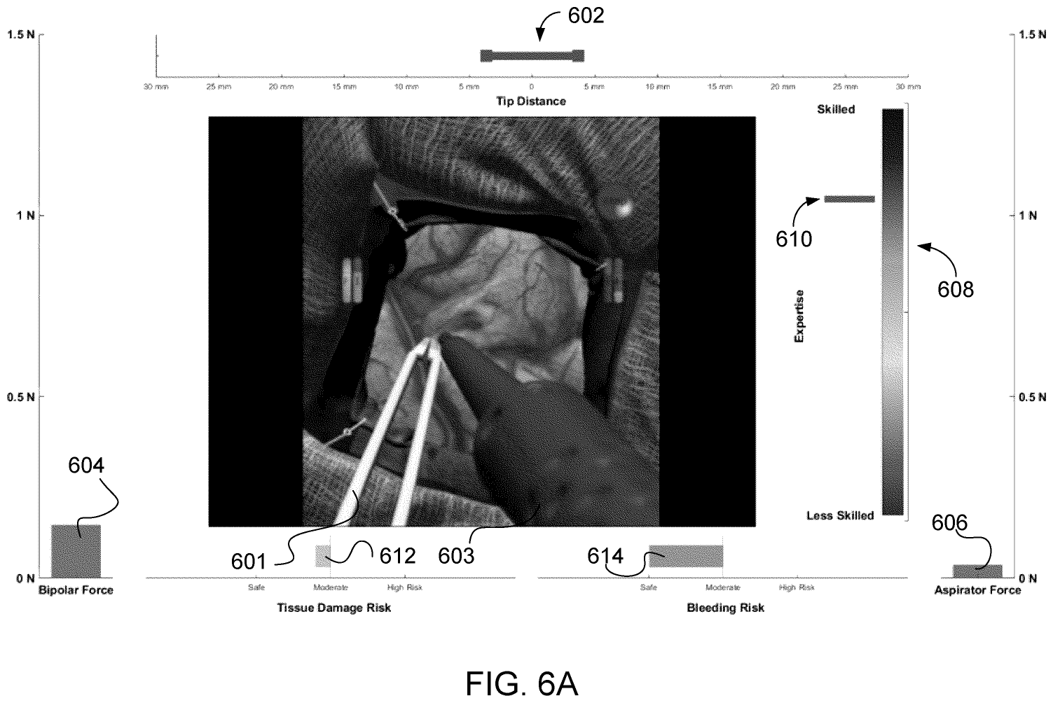

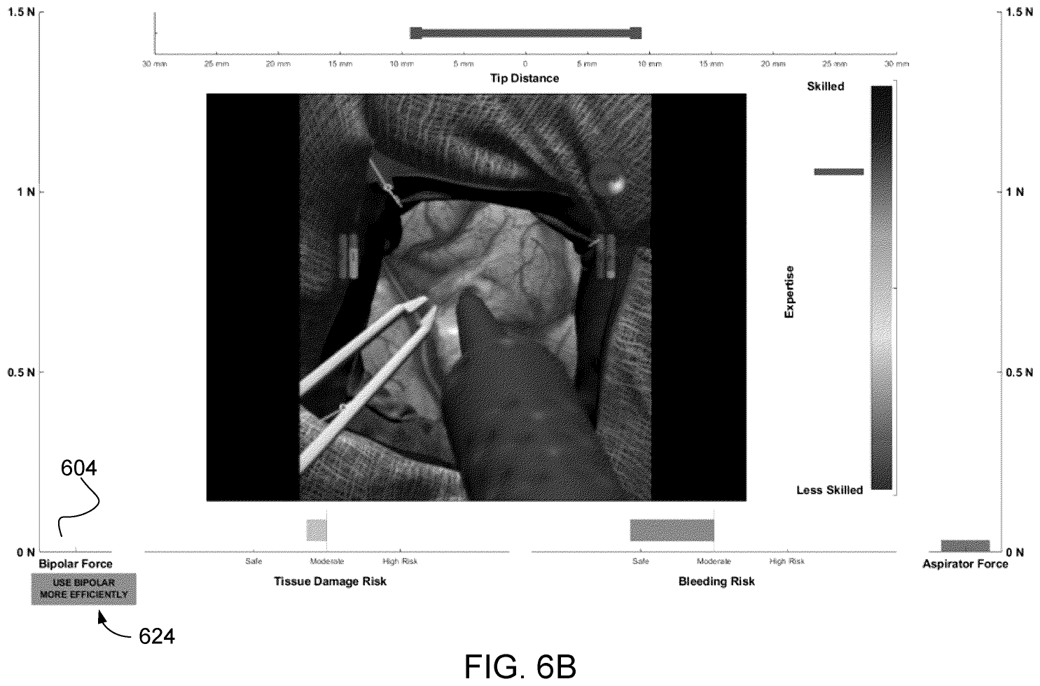

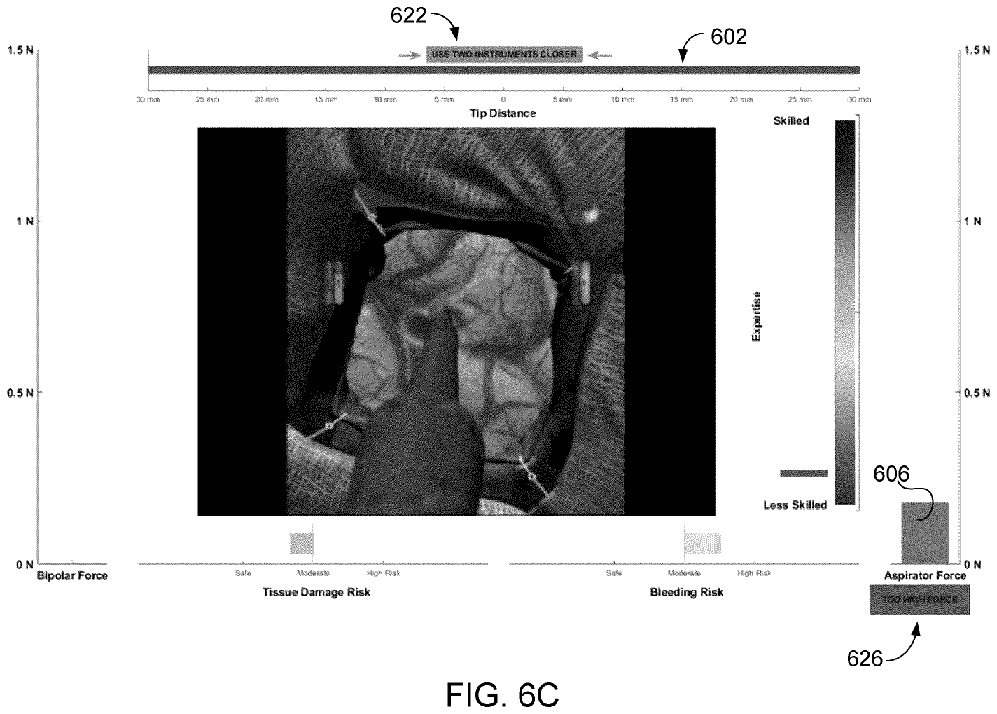

A specific and non-limiting example for evaluating bimanual psychomotor surgical performance using a surgical simulator with haptic feedback is described with reference to FIGS. 6A-6C. In this example, the task performed is a virtual reality tumor resection task and the surgical simulator is used to simulate neurosurgical procedures on a high-fidelity platform configured to recreate the visual, auditory, and haptic experience of resecting human brain tumors. In one embodiment, the procedure is simulated by the surgical simulator using a simulated ultrasonic aspirator in a user's dominant hand and a simulated bipolar forceps in the user's non-dominant hand. The surgical simulator may be used to record time-series data of the user's interaction in the virtual space, such that the simulation system 100 provides extensive information collected from instrument usage from each hand (i.e. dominant and non-dominant). The time interval fluctuates around 20 ms, and interpolation was carried out to provide information in uniform 20 ms intervals. In addition, the 20 ms time interval (50 data points per second) were converted to an average time frame of every 200 ms (only 5 data points per second).

In this example, the algorithms were trained using data from 14 neurosurgeons (expert skill level) and 12 medical students (novice skill level), where each participant performed different simulated tumor resection tasks a number of times. The LSTM network was trained to learn operative surgical expertise from the difference between expert and novice surgical skills, considering the continuous flow of the performance. Each 200 ms was labeled as ‘expert’ for neurosurgeons, and as ‘novice’ for medical students. Every neurosurgeons' data is labeled with ‘1’ and medical students' data is labeled with ‘−1’ for every 200 ms. In one embodiment, use of the LSTM network, as a type of recurrent neural network, may allow for each time point to be evaluated in relation with previous time points, giving the ability to consider sequences in movements.

The raw data provides the location of both instruments 601, 603 at all time points. A task metric of instrument tips separation distance (i.e. the distance between the instrument's tips) 602 is determined from the location of the instruments, at each time point. Other task metrics used in the example are bipolar force 604 and aspirator force 606. In some embodiments, other task metrics related to bimanual technical skills, including, but not limited to, instrument tips separation change as well as force change, velocity, and acceleration of each instrument, may also apply. Speed of bleeding 614 and tissue damage risk 612 are defined as risk metrics. In some embodiments, other risk metrics, including, but not limited to, blood pooling, total blood loss, and blood pooling change, may also apply.

FIG. 6A shows the graphical indicators for the task metrics 602, 604, 606, and risk metrics 612, 614, at a given point in time, as the user is manipulating the instruments 601, 603. The graphical indicators fluctuate with the varying values assigned to the metrics over time. Also displayed is a global skill level 610 compared to a continuous skill scale 608, where the first end (i.e. top) of the scale 608 is for an expert and the second end (i.e. bottom) of the scale 608 is for a novice, the second end opposite to the first end. The global skill level indicator 610 may also vary with time, as the user performs the task.

FIG. 6B shows an example of a guidance message 624, associated with the task metric of bipolar force 604. The graphical indicator for bipolar force 604 indicates that the force currently applied is very weak. Based on a difference between a prediction of the force that should be applied by an expert surgeon at this moment in time of the task and the actual force applied by the user, a message to “use bipolar force more efficiently” is displayed to the user as a form of coaching. This tailored coaching approach is possible because of the labeled time points, which allows the algorithm to consider a time point in the particular task, and the ability to change the output of the algorithm in order to predict the expert's task metric at that time point. An “ideal” bipolar force will differ throughout the task and what is deemed too high or too low at one time point may be suitable at another time point.

FIG. 6C shows an example of another guidance message 622, associated with the task metric of tip distance 602. The graphical indicator for tip distance 602 indicates that the distance is currently very high. Based on a difference between a prediction of the tip distance for an expert surgeon at this moment in time of the task and the actual tip distance of the user, a message to “use two instruments closer” is displayed to the user as a form of coaching. Also shown in FIG. 6C is a guidance message 626 associated with the task metric of aspiration force 606. The user is coached that “too high force” is currently being used with the aspirator tool 603. It will be understood that the examples shown in FIGS. 6A-6C are for illustrative purposes only and many changes may be made, depending on practical implementation.

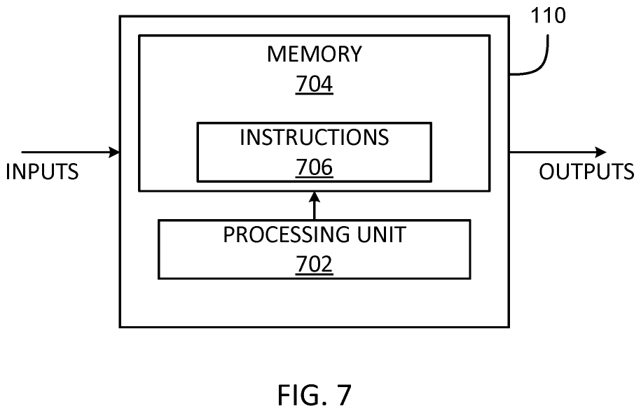

FIG. 7 illustrates an example embodiment for the computing device 110. For simplicity only one computing device 110 is shown but the system may include more computing devices 110 operable to exchange data. The computing devices 110 may be the same or different types of devices.

The computing device 110 comprises a processing unit 702 and a memory 704 which has stored therein computer-executable instructions 706. The processing unit 702 may comprise any suitable devices configured to implement the methods 500, 510, 520 such that instructions 706, when executed by the computing device 110 or other programmable apparatus, may cause the functions/acts/steps performed as part of the methods 500, 510, 520 as described in FIGS. 5A-5C to be executed. The processing unit 702 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory 704 may comprise any suitable known or other machine-readable storage medium. The memory 704 may comprise non-transitory computer readable storage medium, for example, but not limited to, an electronic, magnetic, optical, electromagnetic, infrared, or semiconductor system, apparatus, or device, or any suitable combination of the foregoing. The memory 704 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 704 may comprise any storage means (e.g., devices) suitable for retrievably storing machine-readable instructions 506 executable by processing unit 702.

The methods 500, 510, 520 as described herein may be implemented in a high level procedural or object oriented programming or scripting language, or a combination thereof, to communicate with or assist in the operation of a computer system, for example the computing device 110. Alternatively, the methods 500, 510, 520 may be implemented in assembly or machine language. The language may be a compiled or interpreted language. Program code for implementing the methods and systems may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems may also be considered to be implemented by way of a non-transitory computer-readable storage medium having a computer program stored thereon. The computer program may comprise computer-readable instructions which cause a computer, or more specifically the processing unit 702 of the computing device 110, to operate in a specific and predefined manner to perform the functions described herein, for example those described in the methods 500, 510, 520.

Computer-executable instructions may be in many forms, including program modules, executed by one or more computers or other devices. Generally, program modules include routines, programs, objects, components, data structures, etc., that perform particular tasks or implement particular abstract data types. Typically the functionality of the program modules may be combined or distributed as desired in various embodiments.

The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

The term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described in this document provide non-limiting examples of possible implementations of the present technology. Upon review of the present disclosure, a person of ordinary skill in the art will recognize that changes may be made to the embodiments described herein without departing from the scope of the present technology. For example, more than three algorithms may be running at a time. Yet further modifications could be implemented by a person of ordinary skill in the art in view of the present disclosure, which modifications would be within the scope of the present technology.

1. A method comprising:
obtaining, at a computing device, data at a plurality of time intervals throughout a task performed by a user, the data generated by a control device manipulated by the user while performing the task;
determining, at the computing device, at least one task metric from the data, the at least one task metric associated with the task;
using the at least one task metric to assign, at the computing device, a value to at least one quality assessment metric at each time interval throughout the task based on a progression curve having a novice skill level at a first end of the curve, an expert skill level at a second end of the curve opposite to the first end, and undefined skill levels in between, the at least one quality assessment metric associated with the task; and
displaying in real-time, at the computing device, a first time-varying graphical indicator indicative of the value of the at least one quality assessment metric.

2. The method of claim 1, further comprising:
assigning, at the computing device, a value to at least one risk metric from the data, the at least one risk metric indicative of a negative outcome associated with the task; and
displaying in real time, at the computing device, a second time-varying graphical indicator indicative of the value of the at least one risk metric.

3. The method of claim 2, further comprising displaying, at the computing device, a graphical warning to the user when the value of the at least one risk metric reaches a first threshold.

4. The method of claim 1, further comprising
predicting, at the computing device, the value of the at least one task metric for the expert skill level at a current time point in the task; and
providing, at the computing device, guidance to the user in real-time based on a difference between the predicted value and an actual value of the at least one task metric.

5. The method of claim 4, wherein providing guidance to the user in real-time comprises displaying a guidance message associated with improving the value of the at least one task metric.

6. The method of claim 5, wherein the guidance message is displayed when the difference between the predicted value and the actual value of the at least one task metric is above a second threshold.

7. The method of claim 1, wherein assigning the value to the at least one quality assessment metric comprises applying a regression model that scores the at least one quality assessment metric at every time interval.

8. The method of claim 7, further comprising using a recurrent neural network to implement the regression model.

9. The method of claim 1, wherein displaying in real-time the first time-varying graphical indicator comprises presenting the first time-varying graphical indicator adjacent to a skill level scale representative of the progression curve.

10. The method of claim 1, wherein the task is performed by the user on a virtual simulator.

11. A system comprising:
a processing unit; and
a non-transitory computer-readable medium having stored thereon program instructions executable by the processing unit for:
obtaining data at a plurality of time intervals throughout a task performed by a user, the data generated by a control device manipulated by the user while performing the task;
determining at least one task metric from the data, the at least one task metric associated with the task;
using the at least one task metric to assign a value to at least one quality assessment metric at each time interval throughout the task based on a progression curve having a novice skill level at a first end of the curve, an expert skill level at a second end of the curve opposite to the first end, and undefined skill levels in between, the at least one quality assessment metric associated with the task; and
displaying in real-time a first time-varying graphical indicator indicative of the value of the at least one quality assessment metric.

12. The system of claim 11, wherein the program instructions are further executable for:
assigning a value to at least one risk metric from the data, the at least one risk metric indicative of a negative outcome associated with the task; and
displaying in real time a second time-varying graphical indicator indicative of the value of the at least one risk metric.

13. The system of claim 12, wherein the program instructions are further executable for displaying a graphical warning to the user when the value of the at least one risk metric reaches a first threshold.

14. The system of claim 11, wherein the program instructions are further executable for:
predicting the value of the at least one task metric for the expert skill level at a current time point in the task; and
providing guidance to the user in real-time based on a difference between the predicted value and an actual value of the at least one task metric.

15. The system of claim 14, wherein providing guidance to the user in real-time comprises displaying a guidance message associated with improving the value of the at least one task metric.

16. The system of claim 15, wherein the program instructions are executable for displaying the guidance message when the difference between the predicted value and the actual value of the at least one task metric is above a second threshold.

17. The system of claim 11, wherein assigning the value to the at least one quality assessment metric comprises applying a regression model that scores the at least one quality assessment metric at every time interval.

18. The system of claim 17, wherein a recurrent neural network is used to implement the regression model.

19. The system of claim 11, wherein displaying in real-time the first time-varying graphical indicator comprises presenting the first time-varying graphical indicator adjacent to a skill level scale representative of the progression curve.

20. The system of claim 11, wherein the program instructions are executable for obtaining the data throughout the task performed by the user on a virtual simulator.
